(19) United States
(12) Patent Application Publication
(10) Pub. No.: US 2021/0309810 A1
(43) Pub. Date: Oct. 7, 2021

(54) DOUBLE-CROSSLINKED SELF-HEALING HYDROGEL

(71) Applicant: IUCF-HYU (INDUSTRY-UNIVERSITY COOPERATION FOUNDATION HANYANG UNIVERSITY), Seoul (KR)

(72) Inventors: Kuen Yong LEE, Seoul (KR); Hyun Seung KIM, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 17/219,719

(22) Filed: Mar. 31, 2021

(30) Foreign Application Priority Data
Apr. 3, 2020 (KR) 10-2020-0040991

(51) Int. Cl. C08J 3/075; A61K 47/36; C08L 5/08; C08J 3/24; A61K 35/13

(52) U.S. Cl. CPC C08J 3/075; A61K 47/36; C08L 5/08; C08J 2405/08; A61K 35/13; C08J 2305/08; C08J 3/24

(57) ABSTRACT

Disclosed is a double-crosslinked self-healing hydrogel having excellent mechanical properties and stability and self-healing properties. More particularly, the hydrogel can be injected into the body due to excellent mechanical properties and stability thereof, and thus, can be used as a hydrogel for drug and cell delivery. In addition, the hydrogel can be usefully used as a composition for 3D bioprinters due to self-healing properties thereof

### CROSS-REFERENCE TO RELATED APPLICATION

This application claims priority to and the benefit of Korean Patent Application No. 10-2020-0040991, filed on Apr., 3, 2020, the disclosure of which is incorporated herein by reference in its entirety.

### TECHNICAL FIELD

The present disclosure relates to a self-healing hydrogel having improved mechanical properties and stability due to double crosslinking.

### BACKGROUND ART

Much effort has been made to replace damaged tissues or organs. Artificial replacements, animal-derived non-living tissue or organ transplants have generally been used for patient treatment. However, these materials cannot be a basic solution due to heterogeneous compositions thereof different from original tissues or organs. In recent years, tissue engineering has emerged as an alternative to traditional surgical procedures. For example, cell delivery using in vitro cultured tissues or organs and biomaterials has been applied to tissue engineering approaches, and hydrogels among various types of biomaterials are finding broad applicability.

Hydrogel, also known as hydration gel, is a material that has a network structure wherein water-soluble polymers form three-dimensional crosslinks by physical bonds (hydrogen bonds, van der Waals force, hydrophobic interactions, etc.) or chemical bonds (covalent bonds), and can contain a significant amount of water without dissolution in an aqueous environment. Hydrogel has various chemical compositions and properties because it can be made of various water-soluble polymers. In addition, hydrogel has high biocompatibility due to a high moisture content and physicochemical similarity to the extracellular matrix. Because of these properties, hydrogel has attracted attention as one of very attractive materials for medical and pharmacological applications. In particular, in the case of injecting a hydrogel containing cells, drugs, etc., the self-healing feature of the hydrogel is important to recover from cracking caused by shear force.

Korean Patent No. 10-1865168 discloses an oxidized hyaluronate-based self-healing hydrogel and use thereof for delivering physiologically active substances. However, the self-healing hydrogel has a problem in that the shape or structure thereof cannot be maintained for a long time under physiological conditions due to poor mechanical strength thereof. Further, an ion-crosslinked hydrogel disclosed in Korean Patent No. 10-1704363 exhibits strong mechanical properties and high stability, but has no self-healing properties.

In such a situation, the present inventors have completed a hydrogel having strong mechanical properties, high stability, and self-healing properties.

### DISCLOSURE

### Technical Problem

Therefore, the present disclosure has been made in view of the above problems, and it is one object of the present disclosure to provide a self-healing hydrogel composition capable of double crosslinking, a self-healing double-crosslinkable hydrogel which is prepared using the self-healing hydrogel composition, and a use of the self-healing hydrogel.

### Technical Solution

In accordance with an aspect of the present disclosure, the above and other objects can be accomplished by the provision of a double-crosslinkable hydrogel composition, including: oxidized hyaluronate, glycol chitosan, adipic acid dihydrazide and an alginic acid-grafted hyaluronate modifier,

wherein the oxidized hyaluronate and the glycol chitosan form an imine bond through Schiff base reaction,

the alginic acid-grafted hyaluronate modifier is a structure wherein alginic acid is covalently bonded to a hyaluronate chain, the covalent bond being formed through a linker that allows a covalent bond between a carboxyl group of alginic acid and a carboxyl group of hyaluronate, and

the alginic acid-grafted hyaluronate modifier forms ionic crosslinking.

The present inventors have studied to develop a hydrogel having excellent mechanical properties and stability together with self-healing properties. As a result, it was confirmed that a hydrogel having excellent mechanical properties and stability together with self-healing properties can be prepared by adding hyaluronate, to which alginic acid is bonded, to a hydrogel including oxidized hyaluronate, glycol chitosan and adipic acid dihydrazide and crosslinking the same with a divalent cation.

The term “hydrogel” used in the present specification refers to a three-dimensional structure of a hydrophilic polymer that holds a sufficient amount of moisture, and the term “double crosslinkable hydrogel” refers to a hydrogel in a state in which primary and secondary networks can be formed or have been formed among components constituting the hydrogel. The term “double crosslinkable hydrogel composition” refers to a composition used to prepare a double crosslinkable hydrogel.

In accordance with one embodiment of the present disclosure, the double crosslinkable hydrogel may be a hydrogel wherein oxidized hyaluronate (OHA) and glycol chitosan (GC) form a primary network by an imine bond through Schiff base reaction, and an alginic acid-grafted hyaluronate modifier forms ionic crosslinking by an ionic crosslinking agent to form a secondary network.

The properties of the double crosslinked hydrogel according to the present disclosure may be adjusted depending upon a weight ratio of components constituting the hydrogel, as shown in the following examples. For example, a weight ratio of the oxidized hyaluronate to the glycol chitosan to the adipic acid dihydrazide to the alginic acid-grafted hyaluronate modifier may be 1:1:0.1:0.1 to 5:1:1.5:1.5, preferably 1:1:0.1:0.1 to 3:1:0.8:0.8, most preferably 2:1:0.3:0.3. The weight ratios of the components may mean respective total weight ratios of the oxidized hyaluronate, the glycol chitosan, the adipic acid dihydrazide and the alginic acid-grafted hyaluronate modifier included in the hydrogel composition.

In accordance with one embodiment of the present disclosure, an oxidation degree of the oxidized hyaluronate may be 20% to 80%, preferably 30% to 60%, most preferably 50%.

The term “alginic acid-grafted hyaluronate modifier” used in the present specification means a hyaluronate bonded to alginic acid through a linker capable of forming a covalent bond. In the present specification, the term “alginic acid-grafted hyaluronate modifier” may be referred to as “alginic acid-grafted hyaluronate”, “hyaluronate modifier”, “modifier”, or the like, and these terms have the same meaning.

In accordance with an embodiment of the present disclosure, the covalent bond between alginic acid and hyaluronate of the present disclosure is formed through a linker capable of forming a covalent bond with a carboxyl group of an alginic acid and a carboxyl group of hyaluronate. The linker allowing a covalent bond between hyaluronate and alginic acid may be any linkers having two or more functional groups which are capable of reacting with a carboxylic acid functional group to form a covalent bond, known in the art without specific limitation.

In the present disclosure, the linker may be selected from the group consisting of adipic acid dihydrazide, diamine, divinyl sulfone, 1,4-butanediol diglycidyl ether (BDDE), glutaraldehyde, carbodiimide, hydroxysuccinimide, imidoester, maleimide, haloacetyl, disulfide, hydroazide and alkoxyamine, and is preferably adipic acid dihydrazide.

By using the alginic acid-grafted hyaluronate modifier of the present disclosure, a hydrogel may be simply and easily prepared through ionic crosslinking. Existing hyaluronate hydrogels have be prepared using a chemical crosslinking agent. However, the alginic acid-grafted hyaluronate modifier included in the hydrogel composition of the present disclosure may easily cause ionic crosslinking reaction through addition of divalent cations, such as, for example, Ca²⁺, Ba²⁺, Cu²⁺, Fe²⁺, and Mg²⁺, due to the crosslinking feature of alginic acid introduced into hyaluronate. The use of divalent cations is important in that the possibility that a chemical crosslinking agent induces side effects such as immune or inflammatory reactions in the body is excluded. Thus, the hydrogel composition of the present disclosure may additionally include an ionic crosslinking agent.

In accordance with one embodiment of the present disclosure, a weight ratio of alginic acid to hyaluronate in the alginic acid-grafted hyaluronate modifier may be 10:1 to 1:10, preferably 5:1 to 1:8, most preferably 1:1.

In accordance with one embodiment of the present disclosure, a preferred molecular weight of alginic acid that may be used to prepare the alginic acid-grafted hyaluronate modifier may be 20,000 to 300,0000 g/mol, and a preferred molecular weight of hyaluronate that may be used to prepare the alginic acid-grafted hyaluronate modifier may be 10,000 to 2,000,0000 g/mol.

The double crosslinkable hydrogel composition according to the present disclosure may be used to prepare a double crosslinkable hydrogel. In particular, a mixed solution of glycol chitosan and adipic acid dihydrazide is prepared, and a mixed solution of an oxidized hyaluronate and an alginic acid-grafted hyaluronate modifier is prepared, followed by mixing both the mixed solutions to prepare a self-healing double crosslinkable hydrogel. An ionic crosslinking agent is added to the self-healing double crosslinked hydrogel, thereby preparing a double crosslinked hydrogel.

The present inventors confirmed that, when an alginic acid-grafted hyaluronate modifier was added to a hydrogel including oxidized hyaluronate, glycol chitosan and adipic acid dihydrazide, the mechanical properties and stability of the hydrogel were improved (FIGS. 1 and 3), and the self-healing properties thereof were maintained (FIGS. 5 and 7). However, it was confirmed that when the weight of the alginic acid-grafted hyaluronate modifier relative to the total weight of the hydrogel composition exceeds 0.3%(w/w), the self-healing properties of the hydrogel were decreased (FIG. 5).

Another aspect of the present disclosure provides a composition for three-dimensional bioprinting including the double crosslinkable hydrogel composition.

The composition for three-dimensional bioprinting of the present disclosure refers to a material that can be used as an ink for three-dimensional bioprinters, and the double crosslinkable hydrogel composition of the present disclosure exhibits self-healing properties, when prepared as hydrogel, due to adipic acid dihydrazide included therein. The hydrogel having self-healing properties may recover cracking caused by shear forces when output by a three-dimensional bioprinter.

The present inventors printed a structure using the double crosslinkable hydrogel composition according to the present disclosure as an ink for a bioprinter, and then additionally crosslinked the structure by immersing the same in a solution containing calcium ions. As a result, it was confirmed that the shape of the structure was well maintained (FIG. 8).

Still another aspect of the present disclosure provides a method of preparing the double crosslinked hydrogel including the following steps:

(a) a step of mixing an oxidized hyaluronate solution, a glycol chitosan solution, an adipic acid dihydrazide solution and an alginic acid-grafted hyaluronate modifier solution to prepare a hydrogel; and

(b) a step of treating the hydrogel with a divalent cation or a salt thereof.

In the present disclosure, the oxidized hyaluronate solution, the glycol chitosan solution, the adipic acid dihydrazide solution and the alginic acid-grafted hyaluronate modifier solution of step (a) are substantially the same as those described with regard to the double crosslinked hydrogel composition, and thus, the description thereof is omitted to avoid excessive complexity of the present specification.

In accordance with one embodiment of the present disclosure, the hydrogel of step (a) may be prepared by respectively mixing an oxidized hyaluronate solution, a glycol chitosan solution, an adipic acid dihydrazide solution and an alginic acid-grafted hyaluronate modifier solution. In addition, referring to FIG. 1, the hydrogel may be prepared by mixing a mixed solution of glycol chitosan and adipic acid dihydrazide; and a mixed solution of an oxidized hyaluronate and an alginic acid-grafted hyaluronate modifier.

In accordance with one embodiment of the present disclosure, step (b) of treating the hydrogel with a divalent cation or a salt thereof may be carried out for 10 seconds to 300 seconds, preferably 10 seconds to 200 seconds, more preferably 10 seconds to 120 seconds.

The present inventors confirmed the mechanical properties of the double crosslinked hydrogel according to a treatment time with calcium ions after mixing an oxidized hyaluronate solution, a glycol chitosan solution, an adipic acid dihydrazide solution and an alginic acid-grafted hyaluronate modifier solution. As a result, it was confirmed that there was no significant difference in the mechanical properties of the double crosslinked hydrogel when a treatment time with calcium ions was 1 minute or more (FIG. 4).

Still another aspect of the present disclosure provides a double crosslinked hydrogel prepared by the method and a drug delivery system including the double crosslinked hydrogel.

The drug delivery system according to the present disclosure may be manufactured by a method including the following steps:

(a) a step of mixing oxidized hyaluronate solution, glycol chitosan solution, adipic acid dihydrazide solution, and an alginic acid-grafted hyaluronate modifier solution with a target drug to be delivered to prepare a hydrogel; and

(b) a step of adding divalent cations to the hydrogel to manufacture a double crosslinked hydrogel drug delivery system.

The term “drug” used in the present specification refers to a material capable of exerting a desired useful effect upon introduction into the body and may be selected from compounds, proteins, peptides, nucleic acids, saccharides, extracellular matrix substances, and cells.

In the present disclosure, the compounds may be antibiotics, anticancer agents, analgesics, anti-inflammatory agents, antiviral agents, antibacterial agents, or the like, and the proteins and the peptides may be selected from the group consisting of hormones, cytokines, enzymes, antibodies, growth factors, transcriptional regulators, blood factors, vaccines, structural proteins, ligand proteins and receptors, cell surface antigens, and receptor antagonists. The nucleic acids may be oligonucleotides, DNA, RNA or PNA, and the saccharides may be selected from the group consisting of heparin, heparan sulfate, keratan sulfate, dermatan sulfate, chondroitin sulfate, and hyaluronate.

In addition, the extracellular matrix substances may be selected from the group consisting of collagen, fibronectin, gelatin, elastin, osteocalcin, fibrinogen, fibromodulin, tenascin, laminin, osteopontin, osteonectin, perlecan, versican, von Willebrand factor and vitronectin, and the cells may be selected from the group consisting of fibroblasts, vascular endothelial cells, smooth muscle cells, nerve cells, bone cells, skin cells, chondrocytes, Schwann cells, and stem cells.

### Advantageous Effects

A double-crosslinked self-healing hydrogel according to an embodiment of the present disclosure has excellent mechanical properties and stability and self-healing properties, thus being capable of being usefully used as a hydrogel for drug and cell delivery and a composition for 3D bioprinters.

### Description of Drawings

The above and other objects, features and other advantages of the present disclosure will be more clearly understood from the following detailed description taken in conjunction with the accompanying drawings, in which:

FIG. 1 schematically illustrates a preparation process of a double-crosslinked self-healing hydrogel according to an embodiment of the present disclosure;

FIG. 2 illustrates confirmation results of changes in the weight and diameter of a double-crosslinked self-healing hydrogel over time;

FIG. 3 illustrates confirmation results of the mechanical strength of double-crosslinked self-healing hydrogels that include alginic acid-grafted hyaluronate modifiers (HAH) at various concentrations;

FIG. 4 illustrates confirmation results of the mechanical strength of double-crosslinked self-healing hydrogels according to treatment times with calcium ions;

FIG. 5 illustrates confirmation results of the self-healing properties of double-crosslinked self-healing hydrogels that include alginic acid-grafted hyaluronate modifiers (HAH) at various concentrations;

FIG. 6 illustrates a confirmation result of the self-healing properties of a double-crosslinked self-healing hydrogel;

FIG. 7 illustrates a confirmation result of the self-healing properties of a double-crosslinked self-healing hydrogel disk after cutting and bonding the double-crosslinked self-healing hydrogel disk;

FIG. 8 illustrates confirmation results of double-crosslinked self-healing hydrogel structures that were printed using a bio-ink; and

FIG. 9 illustrates confirmation results of, after printing structures of double-crosslinked self-healing hydrogel including cells using a bio-ink, cell viability in the structures.

### MODES OF THE INVENTION

Hereinafter, one or more embodiments are described in more detail through examples. However, these examples are for illustrative purposes only and the scope of the present disclosure is not limited to these examples.

### EXAMPLE 1

### Preparation of Double-Crosslinked Self-Healing Hydrogel

1-1. Preparation of Oxidized Hyaluronate and Glycol Chitosan

Sodium hyaluronate (MW 2,500,000) was purchased from Lifecore, and glycol chitosan (GC; MW 50,000, Sigma Aldrich) was provided from Wako.

1 g of hyaluronate (HA) was dissolved in 90 ml of distilled water. 0.26735 g of sodium periodate was dissolved in 10 ml of distilled water, followed by stirring the same. The sodium periodate solution was added to an HA solution under dark conditions. Next, the mixture was stirred for 24 hours. This solution was purified through dialysis using distilled water containing sodium chloride for 3 days. After dialysis, the solution was treated with activated carbon, followed by filtration (pore size: 0.22 μm). This filtrate was freeze-dried to obtain an oxidized hyaluronate (hereinafter referred to as OHA) having an oxidation degree of 50%. 1 g of glycol chitosan (GC) was dissolved in 100 ml of distilled water, and purified in the same manner as in the method described above.

1-2. Preparation of Alginic Acid-Grafted Hyaluronate Modifier

For introduction of an amine group, 1-ethyl-3-(dimethylaminopropyl)carbodiimide (EDC; Sigma Aldrich), N-hydroxysulfosuccinimide (Sulfo-NHS; Thermo) and adipic acid dihydrazide were added to 1 g of hyaluronate (molecular weight 1,000,000; Humedix), followed by actively stirring the same for 20 hours to synthesize NH₂-hyaluronate. Next, the resultant solution was freeze-dried.

Next, alginic acid (molecular weight 200,000-300,000; FMC Biopolymer) was combined with NH₂-hyaluronate through carbodiimide chemistry to obtain an alginic acid-grafted hyaluronate modifier (hyaluronate-g-alginate; hereinafter referred to as HAH).

1-3. Preparation of Self-Healing Double-Crosslinkable Hydrogel

1% by weight of glycol chitosan (GC) (relative to the total weight of hydrogel) and 0.3% by weight of adipic acid dihydrazide (ADH; Sigma-Aldrich) were dissolved in distilled water, and, separately, 2% by weight of oxidized hyaluronate (OHA) and 0.3% by weight of HAH were dissolved in distilled water. Next, both the solutions were mixed to prepare a hydrogel. This hydrogel preparation method was adopted because oxidized hyaluronate (OHA) can immediately react with GC&ADH to form a gel, whereas HAH can form an ionic bond with glycol chitosan (GC). Next, CaSO₄ slurry was added in an amount (in an amount wherein 0.42 g of CaSO₄ was mixed with 1 g of alginic acid) of being dependent upon the content of alginic acid in the hydrogel to the hydrogel, thereby preparing a double crosslinked self-healing hydrogel (OHA-GC-ADH-HAH). Calcium ions serve to induce crosslinking among HAH.

As comparative examples, an existing self-healing hydrogel (OHA-GC-ADH), composed of glycol chitosan (GC), adipic acid dihydrazide (ADH) and an oxidized hyaluronate (OHA), and hydrogel (OHA-GC-ADH-ALG), to which alginic acid (0.3% by weight) and calcium ions were added, were respectively prepared.

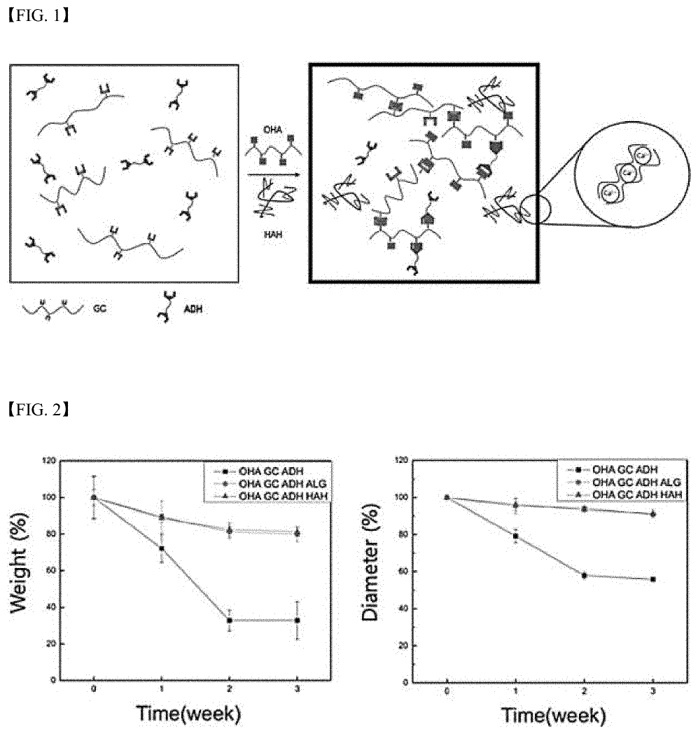

FIG. 1 schematically illustrates a process of preparing a double-crosslinked self-healing hydrogel of the present disclosure.

### EXAMPLE 2

### Property Confirmation of Double Crosslinkable Self-Healing Hydrogel

2-1. Confirmation of Change in Volume and Weight of Double-Crosslinked Self-Healing Hydrogel

The double-crosslinked self-healing hydrogel prepared in Example 1-3 was manufactured in a circular shape. Changes in the weight and diameter of the hydrogel were observed over time while storing at room temperature.

As a result, changes in the weight and size of the double-crosslinked self-healing hydrogel (OHA-GC-ADH-HAH and OHA-GC-ADH-ALG) over time were little, compared to the existing self-healing hydrogel (OHA-GC-ADH). This result indicates that the stability of hydrogel significantly increases due to double crosslinking (FIG. 2).

2-2. Confirmation of Mechanical Strength of Self-Healing Double-Crosslinkable Hydrogel

Double-crosslinked self-healing hydrogels having different HAH concentrations were prepared, and the mechanical strength thereof was measured. For the measurement, a rotary flowmeter equipped with a cone and a plate fixture (plate diameter: 20 mm, cone angle: 4°) was used, and the temperature was kept constant at 25° C.

As measurement results, it was confirmed that the mechanical strength of a self-healing hydrogel (OHA-GC-ADH) increased in proportion to the concentration of HAH when HAH was added to the hydrogel, and the mechanical strength increased by about two times when HAH was added in an amount of 0.3% by weight. In addition, it was confirmed that mechanical strength was further improved due to double crosslinking when calcium ions were added (FIG. 3).

2-3. Optimization of Self-Healing Double Crosslinked Hydrogel

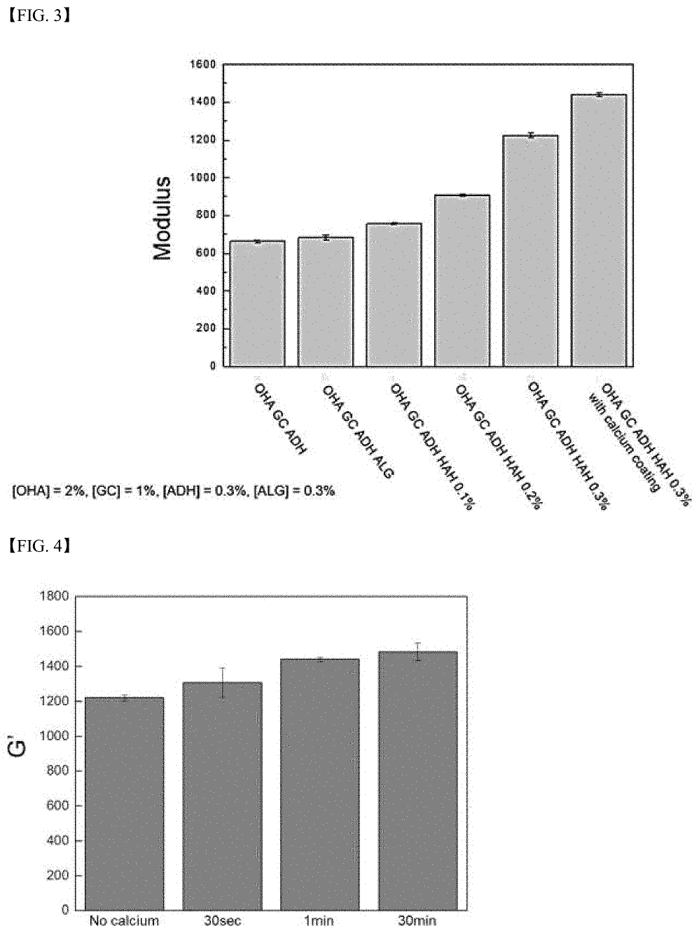

Experiments were carried out as in Example 1-3, except that double-crosslinked self-healing hydrogels were prepared while varying a treatment time with calcium ions. The prepared hydrogels were subjected to mechanical strength measurement. As measurement results, it was confirmed that there was no significant difference in mechanical strength of the hydrogels when a treatment time with calcium ions exceeded 1 minute (FIG. 4). An optimal treatment time with calcium ions was determined to be 1 minute because a non-uniform hydrogel may be rather formed due to internal penetration of calcium, not hydrogel surface coating effect, with increasing treatment time with calcium ions.

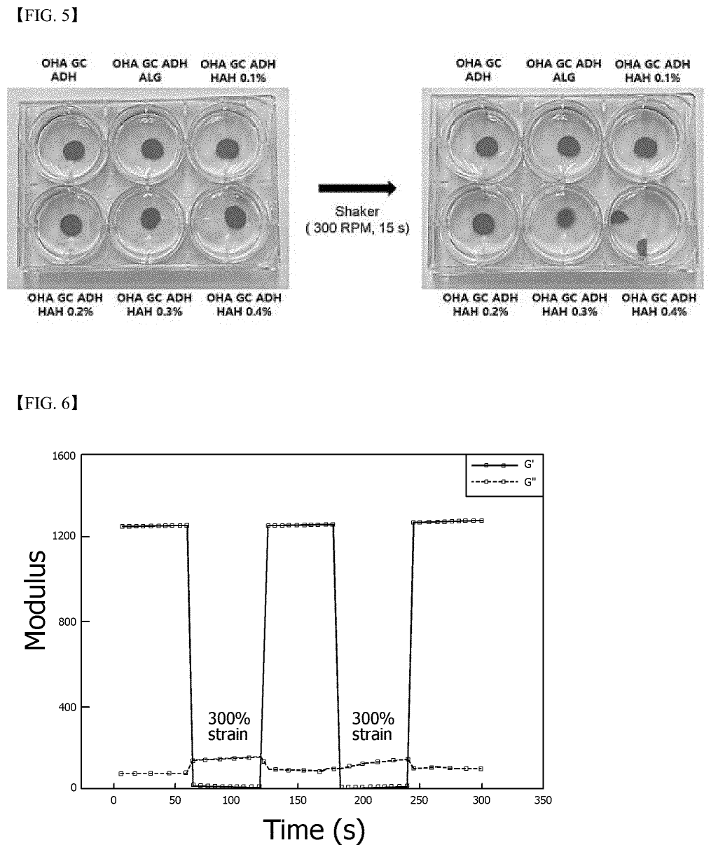

In addition, double-crosslinked self-healing hydrogel disks containing HAH at various concentrations were manufactured and cut, followed by bonding for 30 minutes. These double-crosslinked self-healing hydrogel disks were shaken with a mixer (300 RPM, 15 seconds) to verify self-healing. As results, it was confirmed that self-healing properties of the hydrogel were decreased when the concentration of HAH included in the hydrogel was 0.4% by weight or more (FIG. 5). Thus, the concentration of HAH was determined to be 0.3% by weight at which the self-healing properties of the hydrogel were not affected while increasing the mechanical properties thereof.

2-4. Confirmation of Self-Healing Properties of Double Crosslinkable Hydrogel

The self-healing properties of the self-healing hydrogel prepared in Example 1-3 were evaluated while alternating a strain value to 1% and 300% using a rotary flowmeter. Here, the strain value was applied for 1 minute at each of 1% and 300%. As evaluation results, it was confirmed that, when the structure of the self-healing double-crosslinkable hydrogel (OHA-GC-ADH-HAH, [OHA]=2% by weight, [GC]=1% by weight, [ADH]=0.3% by weight and [HAH]=0.3% by weight) was deformed and destroyed, and then the applied force was removed, the original properties of the self-healing double crosslinked hydrogel were recovered (FIG. 6).

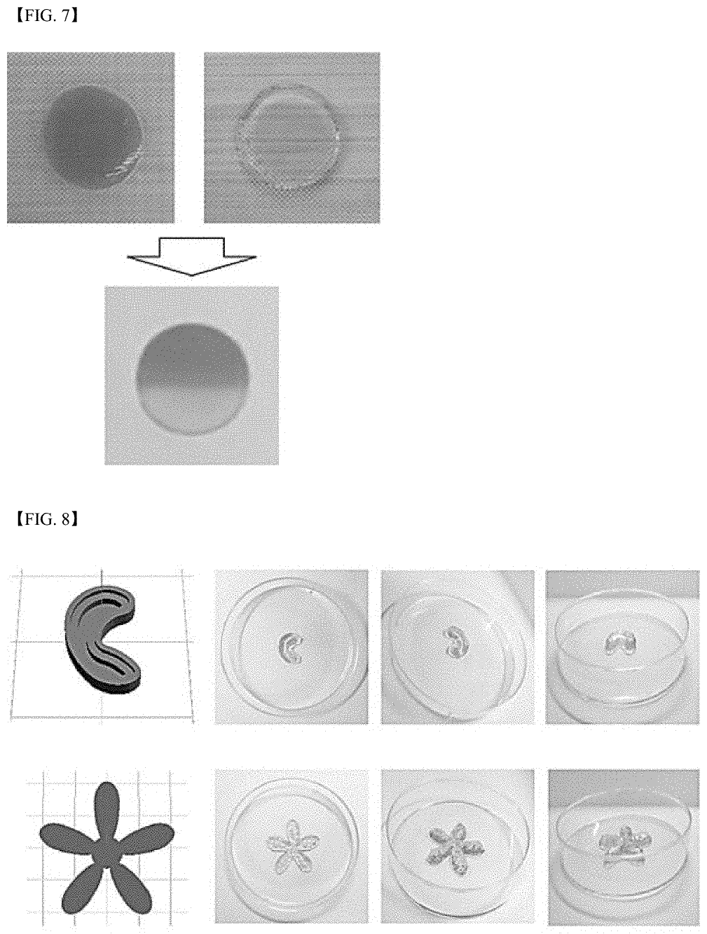

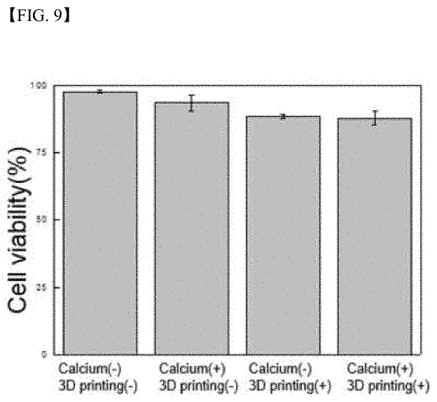

In addition, two different self-healing double-crosslinkable hydrogel disks (OHA-GC-ADH-HAH, [OHA]=2% by weight, [GC]=1% by weight, [ADH]=0.3% by weight and [HAH]=0.3% by weight) with or without a pigment (rhodamine B) were manufactured. Each of the disks was cut into two pieces and optionally fitted together. After 30 minutes, it was visually checked whether the disks were bonded. As results, it was confirmed that an interface of each of the disks disappeared and rhodamine B moved, indicating that the pieces of each of the cut disks were bonded to each other (FIG. 7).

### Example 3

### Use of Double-Crosslinked Self-Healing Hydrogel

3-1. Ink for 3D Bioprinters

The hydrogel solution excluding calcium ions of Example 1-3 was printed into various structure shapes using an ink for 3D bioprinters. The printed structures were immersed in a solution containing calcium ions for 1 minute to be crosslinked. Next, the structure was washed with PBS. As results, it was confirmed that the hydrogel solution satisfactorily served as a bio-ink to be satisfactorily printed in a designed structure shape (FIG. 8).

3-2. Cell Delivery System

ATDC5 cells were mixed at a concentration of 10⁷/ml with a hydrogel solution excluding calcium ions. The mixture was used as an ink for bioprinters and printed in the shape of a structure. The printed structure was immersed in a solution containing calcium ions for 1 minute to be crosslinked, and was washed with PBS. Next, the survival rate of cells included in the structure was investigated using Live/DEAD Viability/Cytotoxicity Kit (Invitrogen).

As results, it was confirmed that cell viability was not significantly affected by the secondary crosslinking by calcium ions, and the 3D bioprinting, indicating that the above processes hardly affected cells.

What is claimed is:

1. A double-crosslinkable hydrogel composition, comprising:
oxidized hyaluronate, glycol chitosan, adipic acid dihydrazide and an alginic acid-grafted hyaluronate modifier,
wherein the oxidized hyaluronate and the glycol chitosan form an imine bond through Schiff base reaction,
the alginic acid-grafted hyaluronate modifier is a structure wherein alginic acid is covalently bonded to a hyaluronate chain, the covalent bond being formed through a linker that allows a covalent bond between a carboxyl group of alginic acid and a carboxyl group of hyaluronate, and
the alginic acid-grafted hyaluronate modifier forms ionic crosslinking.

2. The double-crosslinkable hydrogel composition according to claim 1, wherein a weight ratio of oxidized hyaluronate to glycol chitosan to adipic acid dihydrazide to an alginic acid-grafted hyaluronate modifier is 1:1:0.1:0.1 to 5:1:1.5:1.5.

3. The double-crosslinkable hydrogel composition according to claim 1, wherein an oxidation degree of the oxidized hyaluronate is 20% to 80%.

4. The double-crosslinkable hydrogel composition according to claim 1, wherein a weight ratio of alginic acid to hyaluronate in the alginic acid-grafted hyaluronate modifier is 10:1 to 1:10.

5. The double-crosslinkable hydrogel composition according to claim 1, wherein the linker of the alginic acid-grafted hyaluronate modifier is selected from the group consisting of adipic acid dihydrazide, diamine, divinyl sulfone, 1,4-butanediol diglycidyl ether (BDDE), glutaraldehyde, carbodiimide, hydroxysuccinimide, imidoester, maleimide, haloacetyl, disulfide, hydroazide and alkoxyamine.

6. The double-crosslinkable hydrogel composition according to claim 1, wherein the hydrogel composition further comprises an ionic crosslinking agent.

7. The double-crosslinkable hydrogel composition according to claim 6, wherein the ionic crosslinking agent is a divalent cation or a salt thereof.

8. The double-crosslinkable hydrogel composition according to claim 7, wherein the divalent cation is selected from the group consisting of calcium, barium, copper, iron and magnesium ions.

9. A composition for three-dimensional bioprinting, comprising the double crosslinkable hydrogel composition according to claim 1.

10. A method of preparing a double crosslinked hydrogel, the method comprising:
mixing an oxidized hyaluronate solution, a glycol chitosan solution, an adipic acid dihydrazide solution and an alginic acid-grafted hyaluronate modifier solution to prepare a hydrogel; and
treating the hydrogel with a divalent cation or a salt thereof,
wherein the alginic acid-grafted hyaluronate modifier is a structure wherein alginic acid is covalently bonded to a hyaluronate chain, the covalent bond being formed through a linker that allows a covalent bond between a carboxyl group of alginic acid and a carboxyl group of hyaluronate.

11. The method according to claim 10, wherein a weight ratio of the oxidized hyaluronate to the glycol chitosan to the adipic acid dihydrazide to the alginic acid-grafted hyaluronate modifier in the hydrogel of the mixing is 1:1:0.1:0.1 to 5:1:1.5:1.5.

12. The method according to claim 10, wherein treatment with the divalent cation or the salt is carried out for 20 seconds to 300 seconds.

13. The method according to claim 12, wherein the divalent cation is selected from the group consisting of calcium ions, barium ions, and magnesium ions.

14. A double crosslinked hydrogel prepared by the method of claim 10.

15. A drug delivery system comprising the double crosslinked hydrogel of claim 14.

16. The drug delivery system according to claim 15, wherein the drug is selected from the group consisting of compounds, proteins, peptides, nucleic acids, saccharides, and cells.
